(19) United States
(12) Patent Application Publication
(10) Pub. No.: US 2005/0159615 A1
(43) Pub. Date: Jul. 21, 2005

(54) Process for preparation of statins with high syn to anti ratio

(75) Inventors: Revital Lifshitz-Liron, Herzlia (IL); Nurit Perlman, Kfar Saba (IL)

Correspondence Address: KENYON & KENYON, ONE BROADWAY, NEW YORK, NY, 10004, US

(73) Assignee: Entire Interest.

(21) Appl. No.: 11/020,834

(22) Filed: Dec. 23, 2004

Related U.S. Application Data
(60) Provisional application No. 60/532,458, filed on Dec. 24, 2003.
(60) Provisional application No. 60/547,715, filed on Feb. 24, 2004.

(51) Int. Cl. C07C 69/66

(52) U.S. Cl. 560/179

(57) ABSTRACT

Provided is a process for reduction of statin ketoesters and purification of diol esters of the statins through selective crystallization.

### CROSS-REFERENCE TO RELATED APPLICATIONS

This application claims the benefit of U.S. Provisional Application Nos. 60/532,458 filed on Dec. 24,2003 and 60/547,715 filed on Feb. 24, 2004, the disclosures of which are incorporated by reference in their entirety herein.

### FIELD OF THE INVENTION

The present invention related to reduction of statins and increasing their syn to anti ratio.

### BACKGROUND OF THE INVENTION

The class of drugs called statins are currently the most therapeutically effective drugs available for reducing low-density lipoprotein (LDL) particle concentration in the blood stream of patients at risk for cardiovascular disease and thus, statins are used in the treatment of hypercholesterolemia, hyperlipoproteinemia, and atherosclerosis. A high level of LDL in the bloodstream has been linked to the formation of coronary lesions that obstruct the flow of blood and can rupture and promote thrombosis. Goodman and Gilman, The Pharmacological Basis of Therapeutics, page 879 (9th Ed. 1996).

Statins inhibit cholesterol biosynthesis in humans by competitively inhibiting the 3-hydroxy-3-methyl-glutaryl-coenzyme A (“HMG-CoA”) reductase enzyme. HMG-CoA reductase catalyzes the conversion of HMG to mevalonate, which is the rate determining step in the biosynthesis of cholesterol. Decreased production of cholesterol causes an increase in the number of LDL receptors and corresponding reduction in the concentration of LDL particles in the bloodstream. Reduction in the LDL level in the bloodstream reduces the risk of coronary artery disease. J.A.M.A. 1984, 251, 351-74.

Currently available statins include lovastatin, simvastatin, pravastatin, fluvastatin, cerivastatin and atorvastatin. Lovastatin (disclosed in U.S. Pat. No. 4,231,938) and simvastatin (ZOCOR; disclosed in U.S. Pat. No. 4,444,784 and WO 00/53566) are administered in the lactone form. After absorption, the lactone ring is opened in the liver by chemical or enzymatic hydrolysis, and the active hydroxy acid is generated. Pravastatin (PRAVACHOL; disclosed in U.S. Pat. No. 4,346,227) is administered as the sodium salt. Fluvastatin (LESCOL; disclosed in U.S. Pat. No. 4,739,073) and cerivastatin (disclosed in U.S. Pat. Nos. 5,006,530 and 5,177,080), also administered as the sodium salt, are entirely synthetic compounds that are in part structurally distinct from the fungal derivatives of this class that contain a hexahydronaphthalene ring. Atorvastatin and two new “superstatins,” rosuvastatin and pitavastatin, are administered as calcium salts. The structural formulas of these statins are shown below.

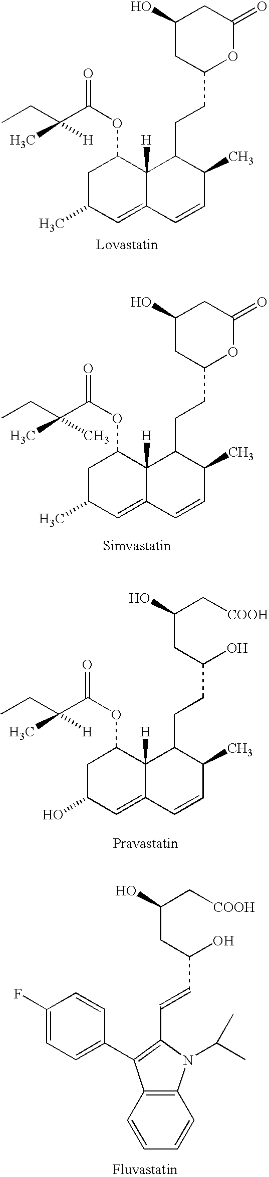

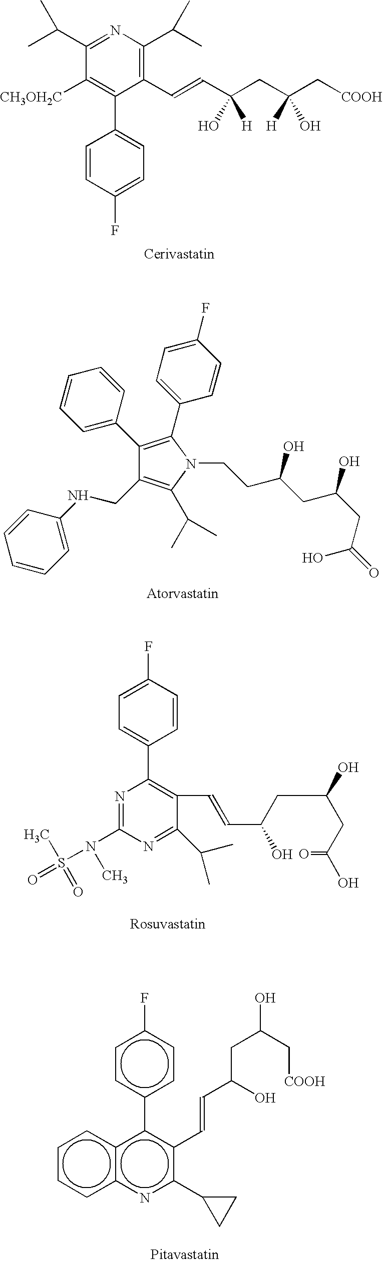

[R*,S*-(E)]-(±)-7-[3-(4-fluorophenyl)-1-(1-methylethyl)-1H-indol-2-yl]-3,5-dihydroxy-6-heptenoic acid is fluvastatin and its structure is depicted above.

A step in the synthesis of statins is reduction of a ketoester to yield the statin. For example, with fluvastatin, in U.S. Pat. No. 5,354,772, a ketoester of fluvastatin is reduced with EtB₃/NaBH₄ to obtain a diol ester. In another patent, U.S. Pat. No. 5,189,164 (EP 0 363 934), a ketoester of fluvastatin is reduced with diethylmethoxyborane to provide fluvastatin. Both these US patents relate to a process of purifying the FLV-diol ester by chromatography only. In U.S. Pat. No. 5,260,440, relating to rosuvastatin and in the U.S. Pat. No. 5,856,336, relating to pitavastatin, the statin-diol esters are also isolated by chromatography. In example 8 of WO 03/004455, 6-dibenzylcarbamoyl-5-hydroxy-3-oxo-hexanoic acid tert-butyl ester is reduced by hydrogenation at a pressure of 25 bar, followed by drying of ethyl acetate to obtain a residue having a syn to anti ratio of 7.6 to 1.

Reduction of a ketoester is also disclosed in Tetrahedron 49, 1997-2010 (1993). In the paper, reduction of a ketoester, which is not a particular statin, is carried out by EtB₃/NaBH₄ or RU-binap to provide a diol ester. In another paper, a ketoester, which is also not any particular statin, is reduced by catecholborane in the optional presence of Rh(PPh₃)Cl. JOC 55, 5190-5192 (1990).

The choice of reducing agents is an important factor in obtaining a statin from its corresponding ketoester since it influences the ratio of syn to anti obtained. The United States Pharmacopeia (USP) provides standards regarding the ratio of syn to anti that is used in a statin formulation. The USP requirements dictate use of a reducing agent that allows obtaining a high syn to anti ratio.

There is a need in the art for reducing agents which may be employed on an industrial scale on a cost effective basis, and which provide a high ratio of syn to anti and overall yield.

The diol ester obtained after reduction is usually not isolated, and is hydrolyzed to obtain a salt. For example, in U.S. Pat. No. 5,003,080, the intermediate ester isn't isolated at all. In one instance however, in Journal of Labeled Compounds & Radiopharmaceuticals vol. XLI, pages 1-7 (1988), a fluvastatin diol ester is obtained from hexane containing 3% isopropanol by volume. (See also TETRAHEDRON, VOL. 53 (31), 10659-10670, 1997)

We have yet found additional ways to increase the Syn to anti ratio of statins through isolation of the diol ester.

### SUMMARY OF THE INVENTION

In one aspect, the present invention provides a process for preparing a statin diol ester having the formula: 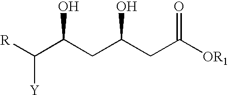
wherein R is an organic radical that is inert to reduction and allows for inhibition of 3-hydroxy-3-methyl-glutaryl-coenzyme A, R₁ is a straight or branched C₁ to C₄ alkyl group, Y is hydrogen or forms a double bond with the R group;
comprising the steps of
a) combining a ketoester of the statin having the formula: 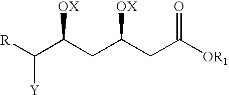
   with a solvent to form a solution;
b) cooling the solution to a temperature of about −50° C. to about −80° C.;
c) combining B-Methoxy-9-BBN with the solution to obtain a reaction mixture, and maintaining the reaction mixture for at least about 30 minutes;
d) combining a source of hydride ions with the reaction mixture, and maintaining the reaction mixture for an additional period of at least about 2 hours;
e) quenching the reaction mixture; and
f) recovering the statin diol-ester,
   wherein at least one X forms a double bond to give a ketone, and at most one X is a hydrogen.

In another aspect, the present invention provides a process for preparing a statin from a statin diol ester having the formula: 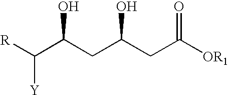
wherein R is an organic radical that is inert to reduction and allows for inhibition of 3-hydroxy-3-methyl-glutaryl-coenzyme A, R₁ is a straight or branched C₁ to C₄ alkyl group, Y is hydrogen or forms a double bond with the R group;
comprising the steps of
a) combining a ketoester of the statin having the formula: 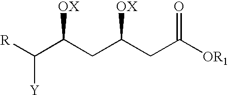
   with a solvent to form a solution;
b) cooling the solution to a temperature of about −50° C. to about −80° C.;
c) combining B-Methoxy-9-BBN with the solution to obtain a reaction mixture, and maintaining the reaction mixture for at least about 30 minutes;
d) combining a source of hydride ions with the reaction mixture, and maintaining the reaction mixture for an additional period of at least about 2 hours;
e) quenching the reaction mixture; and
f) recovering the statin diol-ester,
   wherein at least one X forms a double bond to give a ketone, and at most one X is a hydrogen.

In another aspect, the present invention provides a process preparing a statin from a statin ketoester having the formula: 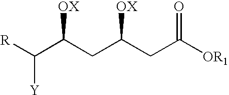
wherein R is an organic radical that is inert to reduction and allows for inhibition of 3-hydroxy-3-methyl-glutaryl-coenzyme A, R₁ is a straight or branched C₁ to C₄ alkyl group, Y is hydrogen or forms a double bond with the R group, at least one X forms a double bond to give a ketone, and at most one X is a hydrogen.
comprising the steps of
a) combining the ketoester of the statin with a solvent to form a solution;
b) cooling the solution to a temperature of about −50° C. to about −80° C.;
c) combining B-Methoxy-9-BBN with the solution to obtain a reaction mixture and maintaining the reaction mixture for at least about 30 minutes;
d) combining a source of the hydride ions to the reaction mixture and maintaining the reaction mixture for an additional period of at least about 2 hours to obtain a diol ester;
e) quenching the reaction mixture;
f) combining the diol ester with NaOH or Ca(OH)₂ and a solvent or a mixture of solvent and water; and
g) recovering the statin free acid, lactone or a pharmaceutically acceptable salt thereof.

In another aspect the present invention provides a process for increasing the syn to anti ratio of fluvastatin diol ester comprising the steps of:
a) dissolving fluvastatin diol ester in a solvent at a temperature of at least about 30° C.;
b) cooling the solution; and
c) recovering the crystallized diol ester.

### DETAILED DESCRIPTION OF THE INVENTION

The present invention provides methods for reduction of a statin ketoester by use of 9-methoxy-9-bora-bicyclo[3.3.1]nonane (B-methoxy-9-BBN) as a reducing agent. Reduction with B-methoxy-9-BBN (BM-9-BBN) provides ideal selectivity. The requirement for fluvastatin diol ester is no more than about 0.8% by area % HPLC of the anti product. The reduction process of the present invention yields about 0.5 to 0.6% anti by area % HPLC, and other crystallization steps yield less than about 0.2% anti by area % HPLC . Additionally, B-methoxy-9-BBN may be used in a molar ratio as low as about 1:1.

The ketoester reduced in the present invention, which is exemplified by fluvastatin, has the following formula: 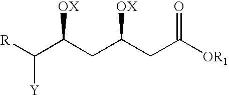
wherein R₁ is a C₁ to C₄ alkyl group (t-butyl preferred), R is an organic radical as described below, Y is a hydrogen or forms a double bond with the R group and at least one of the X's forms a double bond with the carbons being attached to the oxygen to give a ketone, and at most one X is hydrogen. A preferred reaction scheme is illustrated below, where the X closest to the ester forms a ketone and the other X is a hydrogen (alpha ketoester):

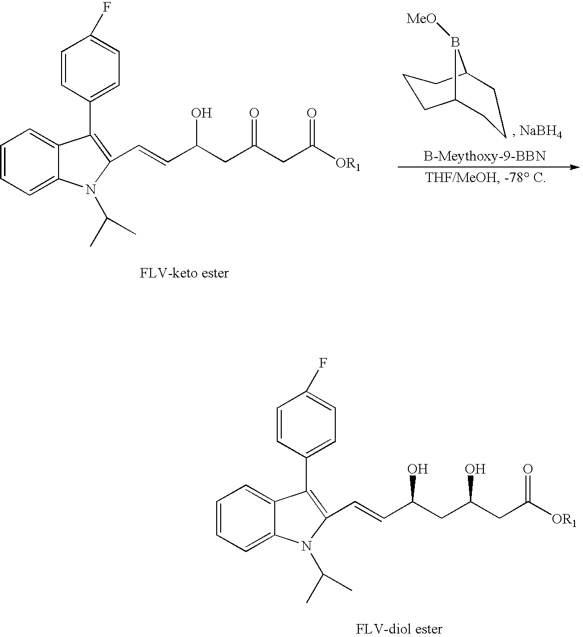

As used herein, R refers to an organic radical that is bonded to the diol pentanoic ester group and is inert to reduction with the reducing agent and allows for therapeutic activity. By inert to reduction it is meant that the reducing agent employed does not reduce the R Group according to the general knowledge of one of skill in the art. Depending on the statin, the R radical can be:
pravastatin: 1,2,6,7,8,8a-Hexahydro-6-hydroxy-2-methyl-8-(2-methyl-1-oxobutoxy)-1-naphthalene ethyl radical.
fluvastatin: 3-(4-fluorophenyl)-1-(1-methylethyl)-1H-indol-2-yl]-ethylene radical.
cerivastatin: 4-(4-fluorophenyl)-5-methoxymethyl)-2,6-bis(1-methylethyl)-3-pyridinyl-ethylene radical.
atorvastatin: 2-(4-fluorophenyl)-5-(1-methylethyl)-3-phenyl-4-[(phenylamino)carbonyl]-1H-pyrrole-ethyl radical
rosuvastatin: [4-(4-fluorophenyl)-6-(1-methylethyl)-2-[methyl(methylsulfonyl)amino]-5-pyrimidinyl]-ethylene radical. pitavastatin: [4′-(4″-fluorophenyl)-2′-cyclopropyl-quinolin-3′-yl]-ethylene radical.

The R radical can also be that of the open ring form, i.e., the dihydroxy acid, of simvastatin or lovastatin. These open ring forms also have a diol pentanoic acid group. As used herein, the terms simvastatin and lovastatin include both the lactone form and the open-ring form, unless otherwise indicated by a formula. When the statin is simvastatin or lovastatin, the R radical is:
simvastatin: 1,2,6,7,8,8a-Hexahydro-2,6-dimethyl-8-(2,2-dimethyl-1-oxobutoxy)-1-naphthalene ethyl radical.
lovastatin: 1,2,6,7,8,8a-Hexahydro-2,6-dimethyl-1-8-(2-methyl-1-oxobutoxy)-1-naphthalene ethyl radical.

The reduction of the statin keto-ester, with B-Methoxy-9-BBN includes combining the statin keto-ester and a solvent; cooling the solution to a temperature of about −50° C. to about −80° C.; adding B-Methoxy-9-BBN and maintaining the reaction mixture for at least about 30 minutes; adding a source of hydride ions and maintaining the reaction mixture for an additional period of at least about 2 hours; adding a quenching agent; and recovering the statin diol-ester. The solvent may include C₁ to C₄ alcohols such as methanol, dipolar solvents such as tetrahydrofuran, C₂ to C₈ ethers cyclic or acyclic, or a mixture thereof. Preferably, the solution is cooled to about −70° C. to about −80° C. An optimum temperature is about −70° C., which allows for greater selectivity. The source of hydride ions may be sodium borohydride, potassium borohydride and lithium borohydride, preferably sodium borohydride. The quenching agent may be any one of hydrogen peroxide, sodium carbonate. −1.5H₂O or NaBO₃.H₂O, preferably hydrogen peroxide. The quenching agent is used for terminating the reaction, by reacting it with the remaining reducing agent.

After quenching the reaction, the diol ester may be recovered from the reaction mixture by adding a C₄ to C₇ ester and water, separating the organic phase from the two-phase system that formed, and removing the solvent by any technique known in the art (such as evaporation).

According to USP pharmacopoeia, the level of anti-isomer should be NMT 0.8% (% area by HPLC according to USP HPLC method). In order to increase the syn to anti isomer ratio the fluvastatin diol ester may be crystallized.

In one embodiment, fluvastatin diol ester in the present invention may be crystallized from the following solvents: C₃ to C₇ ketone such as acetone, C₁ to C₄ alcohol such as ethanol, isopropyl alcohol, 1-propanol, 2-propnaol 1-butanol and 2-butanol, C₃ to C₇ ester other than ethyl acetate such as isopropylacetate, isobutylacetate or methyl acetate, C₁-C₄ ethers other than MTBE (methyl t-butyl ether), and mixtures thereof. The crystallization solvent may also be a mixture of MTBE and C₁ to C₄ alcohols, preferably MTBE and IPA. The crystallization includes the steps of: dissolving the statin diol ester in said solvent at elevated temperature; cooling the solution; and recovering the crystallized fluvastatin diol ester. Preferably, the solvent is selected from the group consisting of: acetone, IPA, isopropylacetate, mixtures thereof and a mixture of IPA/MTBE. The elevated temperature is preferably above about 30° C., more preferably above about 40° C. and most preferably about reflux temperature.

The precipitate obtained may be recovered by conventional techniques such as filtration and concentration. Preferably, the fluvastatin is dissolved at reflux. Seeding may also be used for crystallization.

The fluvastatin diol-ester may also be crystallized by using a solvent and an anti solvent. This comprises the steps of: dissolving the statin diol-ester in a C₃ to C₇ ketone solvent such as acetone, methylethylketone and methyl isopropyl ketone, at elevated temperature; adding a C₅ to C₁₂ saturated hydrocarbon such as cyclic and acyclic heptane and hexane; cooling the solution; and recovering the crystallized diol ester. Preferably, the cooling is at a temperature of from about 10° C. to about 25° C. Preferably, the elevated temperature is the reflux temperature. In one embodiment, a C₁ to C₄ alcohol is used with less than 50% hydrocarbon by volume, more preferably without a hydrocarbon.

The term “anti-solvent” refers to a liquid that, when added to a solution of fluvastatin diol ester in a solvent, induces precipitation of fluvastatin sodium. The anti-solvent may also be in a binary mixture with the solvent when the solution is prepared. Precipitation of fluvastatin diol ester is induced by the anti-solvent when addition of the anti-solvent causes fluvastatin diol ester to precipitate from the solution more rapidly or to a greater extent than fluvastatin diol ester precipitates from a solution containing an equal concentration of fluvastatin in the same solvent when the solution is maintained under the same conditions for the same period of time but without adding the anti-solvent. Precipitation can be perceived visually as a clouding of the solution or formation of distinct particles of fluvastatin diol ester suspended in or on the surface of the solution or collected on the walls or at the bottom of the vessel containing the solution.

The above crystallizations may allow for increasing the syn to anti ratio so that the level of the anti isomer is about 0.2 or less % area by HPLC. Preferably the level of the anti isomer is about 0.04 or less % area by HPLC.

The diol ester may be further converted into a pharmaceutically acceptable salt of the statin or a lactone. In one embodiment, the diol ester obtained is reacted with sodium or calcium hydroxide to obtain the sodium or calcium salt. It is also possible to first obtain the sodium salt by reaction with sodium hydroxide, and then convert the sodium salt to calcium salt by using a source of calcium such as calcium chloride or calcium acetate. The basic hydrolysis of the statin diol-ester may be carried out with one or more equivalents of an alkali metal or alkaline earth metal base such as NaOH or Ca(OH)₂, in organic solvents such as C₁ to C₈ ethers (tetrahydrofuran, IPE), ACN, C₁ to C₄ alcohols (MeOH, EtOH, IPA, propanol, butanol etc.), C₃ to C₈ ketones or esters (acetone, methyl ethyl ketone, methyl isopropyl ketone, ethyl acetate). The hydrolysis may also be carried out with water, a mixture of the above solvents, or a mixture of water and the above solvents, preferably at room temperature or by heating. The lactone may be obtained by treating the acid form with an acid such as HCl.

Pharmaceutical Compositions

Pharmaceutical formulations of the present invention contain pharmaceutically acceptable salts or lactone form of the statins with a high syn to anti ratio. Pharmaceutically acceptable salts include those of alkali and alkaline earth metals, preferably calcium. In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, nateglinide and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets and troches, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Having thus described the invention with reference to particular preferred embodiments and illustrated it with Examples, those in the art can appreciate modifications to the invention as described and illustrated that do not depart from the spirit and scope of the invention as disclosed in the specification. Even though the example illustrates reduction of fluvastatin, the method disclosed herein is generally applicable to the other statins. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications.

### EXAMPLES

### Example 1

### Reduction of FKE-tBu to FDE-tBu

A 1 L triple-jacket reactor, covered with aluminum foil was loaded with FKE-tBu (30 g), THF (CP, 300 ml) and Methanol (CP, 60 ml).

The solution was cooled to (−70° C.) and then BM-9-BBN (1M solution in Hexanes, 71 ml.) was added. The mixture was stirred at (−70° C.) for 30 minutes. Sodium borohydride (2.4 g) was added and the reaction mixture was stirred at (−70° C.) for about 2 hours (monitoring by HPLC for the consumption of FKE-tBu).

A solution of 30% Hydrogen peroxide (48 ml) was added and the reaction mixture was allowed to stir at room temperature for 19.5 hours. The reaction mixture was diluted with EtOAc (150 ml), water (150 ml) and Brine (105 ml). The phases were separated and the organic layer was washed with saturated solution of NaHCO₃ (1×120 ml), saturated solution of Na₂SO₃ (1×120 ml) and Brine (1×120 ml). The organic layer was evaporated under vacuum to dryness.

The obtained solid residue was dissolved in acetone (90 ml) at reflux temperature while the flask was covered with aluminum foil. Then n-Heptane (210 ml) was added at reflux. The mixture was cooled to room temperature and stirred at this temperature for about 18 hours. The product was isolated by filtration under nitrogen atmosphere, washed with n-Heptane (100 ml) and dried at 40° C. in a vacuum oven for 24 hours to obtain 21.9 g (73%) of FDE-tBu crude. First crystallization—Syn:anti—99.0/0.45.

### Example 2

### Crystallization of Crude FLV-diol ester from Acetone and n-Heptane

FDE-tBu crude (syn:anti 99.0:0.45) was dissolved in Acetone (116 ml) at reflux temperature while the flask was covered with aluminum foil. Then n-Heptane (252 ml) was added at reflux. The mixture was cooled to 37° C. during 1 hour, stirred at this temperature for 1 hour and cooled to 20° C. during 1 hour. The obtained slurry was stirred at 20° C. for 15 hours. The product was isolated by filtration under nitrogen atmosphere, washed with n-Heptane (3×66 ml) and dried at 40° C. in a vacuum oven for 24 hours to obtain 18.9 g (90%) of FDE-tBu cryst (syn:anti 99.8:0.17).

### Example 3

### Conversion of FDE-tBu to FLV Na form XIV

Water (56 ml), ACN (200 ml) and FDE-tBu (40 gr) are added to a 1 L stirred reactor. At 25 deg. 7.5 gr of 47% NaOH solution are added and the mixture is heated to 35° C. The mixture becomes clear during the hydrolysis. End of reaction is determined by HPLC (˜3-4 hr). The mixture is then cooled to 25° C. ACN (600 ml) is added to the mixture causing precipitation of FLV Na crystals.

The mixture is stirred for ˜5 hr and then filtered under vacuum.

The wet product is washed with 120 ml of ACN.

The wet product is dried in a vacuum oven at 40° C. to obtain FLV Na form XIV crystals. Yield: 87%

### Example 4

### Conversion of FDE-Me to FLV Na

Fluvastatin-diol methyl ester (3.0 g) was added to solution of NaOH (1 eq.) in water (0.75 ml) and ethanol (7.5 ml). The mixture was heated to reflux and stirred until the raw material wasn't observed by HPLC. After this time 58 ml of MTBE were dripped to the solution during 1.5 hr. Turbidity appeared in the solution, which was cooled slowly to room temperature and stirred over night. The product was isolated by filtration under nitrogen, washed with MTBE (50 ml) and dried at 50° C. in a vacuum oven for 24 hours to obtain 2.21 grams (72.3%) of fluvastatin sodium.

### Example 5

### Conversion of FDE-ME to FLV Na

Fluvastatin-diol-methyl ester (FDE-ME ) (4.0 g) was dissolved in acetone (40 ml). A solution of NaOH (0.38 gr) in MeOH (4 ml) was added and the mixture was stirred at room temperature for 20 hr. The product was isolated by filtration under nitrogen, washed with acetone (20 m) and dried at 50 EC in a vacuum oven for 26 hours to obtain 3.35 gr (82.2%) of fluvastatin sodium.

### Example 6

### Crystallization of Crude FLV-diol ester from IPA

Crude FLV-diol-tert butyl ester (that prepared as mentioned in the reduction procedure with BM-9-BBM) (5.77 gr, Syn:anti—98.6/0.88) was dissolved in IPA (60 ml) by heating to reflux. After 30 minutes, the clear solution was cooled to room temperature and stirred over night. The solution was then concentrated (approximately 17 ml of IVA was evaporated) and stirred at room temperature overnight. The product was isolated by vacuum filtration under nitrogen flow, washed with IPA (30 ml), then dried in vacuum oven at 40° C. for to obtain FLV-diol-tert butyl ester. First crystallization—Syn:anti—98.9/0.61.

### Example 7

### Crystallization of Crude FLV-diol ester from acetone

Crude FLV-diol-t-Butyl ester (4.0 g) was dissolved in acetone (18.5 ml) at reflux temperature. After 45 minutes the clear solution was cooled to room temperature to obtain a massive precipitate. The suspension was diluted with Acetone (10 ml) and the product was isolated by vacuum filtration under nitrogen flow, washed with Acetone (4×10 ml) and dried in a vacuum oven at 50 EC for 24 hours to obtain FLV-diol-t-Butyl ester (1.7 g, 42%). First crystallization—Syn:anti—98.8/0.27; Second crystallization—Syn:anti—99.6/0.04.

### Example 8

### Crystallization of Crude FLV-diol ester from Isobutylacetate

FDE-tBu (3 gr) (Syn:anti—98.6/0.88) was dissolved in Isobutylacetate (48 ml) by reflux.

The solution was cooled to room temperature and stirred over night.

The product was isolated by vacuum filtration, washed with isobutylacetate and dried in vacuum oven at 50° C. for 24 hours to obtain FDE-tBu (1.92 gr, 64% yield). First crystallization—Syn:anti—99.6/0.2.

### Example 9

### Crystallization of Crude FLV-diol ester from IPA and MTBE

FDE-tBu (3 gr, syn:anti 98.6:0.88) was dissolved in IPA (15 ml) by reflux and MTBE (30 ml) was added. The solution was cooled to room temperature and stirred over night. The product was isolated by vacuum filtration, washed with a solution of MTBE:IPA 1:1 v:v (20 ml) and dried in vacuum oven at 40 deg for 24 hours to obtain FDE-tBu (1.5 gr, 51% yield). Syn:anti 99.6:0.20

1. A process for preparing a statin diol ester having the formula: 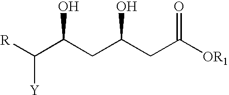
wherein R is an organic radical that is inert to reduction and allows for inhibition of 3-hydroxy-3-methyl-glutaryl-coenzyme A, R₁ is a straight or branched C₁ to C₄ alkyl group, y is hydrogen or forms a double bond with the R group;
comprising the steps of
a) combining a ketoester of the statin having the formula: 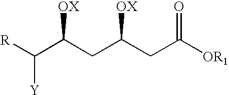
with a solvent to form a solution;
b) cooling the solution to a temperature of about −50° C. to about −80° C.;
c) combining B-Methoxy-9-BBN with the solution to obtain a reaction mixture, and maintaining the reaction mixture for at least about 30 minutes;
d) combining a source of hydride ions with the reaction mixture, and maintaining the reaction mixture for an additional period of at least about 2 hours;
e) quenching the reaction mixture; and
f) recovering the statin diol-ester,
wherein at least one X forms a double bond to give a ketone, and at most one X is a hydrogen.

2. The process of claim 1, wherein the solvent is selected from the group consisting of: C₁ to C₄ alcohol, dipolar aprotic solvent, cyclic or acyclic C₂ to C₈ ether and a mixture thereof.

3. The process of claim 2, wherein the solvent is a mixture of methanol and tetrahydrofuran.

4. The process of claim 1, wherein the solution is cooled to about −70° C. to about −80° C.

5. The process of claim 4, wherein the temperature is about −70° C.

6. The process of claim 1, wherein the source of the hydride ions is selected from the group consisting of: sodium borohydride, potassium borohydride and lithium borohydride.

7. The process of claim 6, wherein the source of the hydride ions is sodium borohydride.

8. The process of claim 1, wherein the quenching agent is selected from the group consisting of hydrogen peroxide, sodium carbonate.1.5H₂O and NaBO₃.H₂O.

9. The process of claim 8, wherein the quenching agent is hydrogen peroxide.

10. The process of claim 1, wherein R is an organic radical that would provide a statin selected from the group consisting of: lovastatin, simvastatin, pravastatin, fluvastatin, cerivastatin, atorvastatin, rosuvastatin and pitavastatin.

11. The process of claim 10, wherein R is an organic radical that would provide fluvastatin.

12. The process of claim 1, wherein the ketoester is an alpha ketoester.

13. A process for preparing a statin from a statin diol ester having the formula: 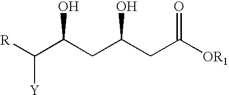
wherein R is an organic radical that is inert to reduction and allows for inhibition of 3-hydroxy-3-methyl-glutaryl-coenzyme A, R₁ is a straight or branched C₁ to C₄ alkyl group, Y is hydrogen or forms a double bond with the R group;
comprising the steps of
a) combining a ketoester of the statin having the formula: 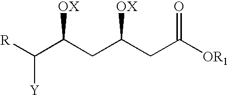
with a solvent to form a solution;
b) cooling the solution to a temperature of about −50° C. to about −80° C.;
c) combining B-Methoxy-9-BBN with the solution to obtain a reaction mixture, and maintaining the reaction mixture for at least about 30 minutes;
d) combining a source of hydride ions with the reaction mixture, and maintaining the reaction mixture for an additional period of at least about 2 hours;
e) quenching the reaction mixture; and
f) recovering the statin diol-ester,
wherein at least one X forms a double bond to give a ketone, and at most one X is a hydrogen.

14. The process of claim 13, wherein the pharmaceutically acceptable salt is calcium salt or sodium salt.

15. A process for preparing a statin from a statin ketoester having the formula: 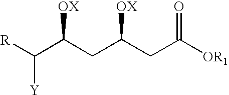
wherein R is an organic radical that is inert to reduction and allows for inhibition of 3-hydroxy-3-methyl-glutaryl-coenzyme A, R₁ is a straight or branched C₁ to C₄ alkyl group, Y is hydrogen or forms a double bond with the R group, at least one X forms a double bond to give a ketone, and at most one X is a hydrogen.
comprising the steps of
a) combining the ketoester of the statin with a solvent to form a solution;
b) cooling the solution to a temperature of about −50° C. to about −80° C.;
c) combining B-Methoxy-9-BBN with the solution to obtain a reaction mixture and maintaining the reaction mixture for at least about 30 minutes;
d) combining a source of the hydride ions to the reaction mixture and maintaining the reaction mixture for an additional period of at least about 2 hours to obtain a diol ester;
e) quenching the reaction mixture;
f) combining the diol ester with NaOH or Ca(OH)₂ and a solvent or a mixture of solvent and water; and
g) recovering the statin free acid, lactone or a pharmaceutically acceptable salt thereof.

16. A process for increasing the syn to anti ratio of fluvastatin diol ester comprising the steps of:
a) dissolving fluvastatin diol ester in a solvent at a temperature of at least about 30° C.;
b) cooling the solution; and
c) recovering the crystallized diol ester.

17. The process of claim 16 wherein the solvent is selected from the group consisting of: C₃ to C₇ ketone, C₁ to C₄ alcohol, C₁ to C₇ ester other than ethyl acetate, C₁-C₈ ethers other than MTBE and mixtures thereof.

18. The process of claim 17, wherein the solvent is a mixture of MTBE and a C₁ to C₄ alcohol.

19. The process of claim 18, wherein the solvent is a mixture of MTBE and IPA.

20. The process of claim 17, wherein the solvent is selected from the group consisting of: acetone, ethanol, isopropyl alcohol, 1-propanol, 2-propnaol, 1-butanol 2-butanol, isopropylacetate, methyl acetate, isobutylacetate and mixtures thereof.

21. The process of claim 20, wherein the solvent is selected from the group consisting of acetone, isopropyl alcohol, isobutylacetate and mixtures thereof.

22. The process of claim 16, wherein the temperature is about reflux temperature.

23. A process for preparing fluvastatin diol ester comprising converting the product of claim of 16 to a fluvastatin free acid, lactone or a pharmaceutically acceptable salt thereof.

24. The process of any of claims 16, wherein the level of the anti isomer is about 0.2 or less % area by HPLC.

25. A process for increasing the syn to anti ratio of fluvastatin comprising the steps of:
a) dissolving fluvastatin diol ester in a C₃-C₇ ketone at a temperature of as least about 30° C;
b) combining a C₅ to C₁₂ saturated hydrocarbon with the solution;
b) cooling the ketone/hydrocarbon mixture; and
c) recovering the crystallized diol ester.

26. The process of claim 25, wherein the C₃-C₇ ketone is selected from the group consisting of acetone, methylethylketone, methyl isopropyl ketone and mixtures thereof.

27. The process of claim 25, wherein the C₅ to C₁₂ saturated hydrocarbon is heptane or hexane.

28. The process of claim 25, wherein the temperature is about reflux temperature.

29. The process of claim 25, wherein the cooling temperature is about 10° C. to about 25° C.

30. The process of claim 25, wherein the process further comprises converting the crystallized diol ester to a fluvastatin free acid, lactone or a pharmaceutically acceptable salt thereof.

31. The process of any of claims 25, wherein the level of the anti isomer is about 0.2 or less % area by HPLC.

32. The process of any of claim 31, wherein the level of the anti isomer is about 0.04 or less % area by HPLC.
